(19) United States
(12) Patent Application Publication
(10) Pub. No.: US 2014/0114266 A1
(43) Pub. Date: Apr. 24, 2014

(54) Ostomy Implant System and Method

(71) Applicant: AMS Research Corporation, (US)

(72) Inventor: Benjamin Y Arcand, Minneapolis, MN (US)

(73) Assignee: AMS RESEARCH CORPORATION, Minnetonka, MN (US)

(21) Appl. No.: 13/657,385

(22) Filed: Oct. 22, 2012

(51) Int. Cl. A61F 5/449; A61F 5/445

(52) U.S. Cl. CPC A61F 5/449; A61F 5/445
USPC 604/345; 604/332

(57) ABSTRACT

Various embodiments of an ostomy implant system are provided. A 3D-structured ostomy implant can adapt to the shape of the interface between the intestine and the abdominal wall. The implant can include a funnel-like device where a funnel neck portion can encapsulate and support the intestine and a larger funnel top can attach to and reinforce the abdominal wall. The implant can be a unitary implant constructed of a plurality of undulating strut members.

### FIELD OF THE INVENTION

The present invention relates generally to surgical methods and apparatus and, more specifically, to an implant system and device adapted to create a continent ostomy and prevent herniation.

### BACKGROUND OF THE INVENTION

An ostomy is a surgical procedure that creates an opening in the abdominal wall for waste products to move out of the body. It is performed when a medical condition is so severe that an ostomy offers a better alternative—e.g., when cancer, trauma, malformations, obstructions, Crohn's disease and other conditions or diseases have required removal of all or a substantial amount of the patient's intestines.

Studies have shown that having ostomy surgery and caring for an ostomy can cause problematic quality of life issues and concerns for patients. The physical aspects of wearing the appliance itself, such as the possible fear of leakage and odor, can lead patients to social isolation. Further, patients often do not seek help once they have been released from the hospital. The psychological impact on body image, sexual activity, coping and adjustment is significant. Poor psychosocial adjustment has been linked to increased depression and death in the ostomate population.

One of the most difficult outcomes of an ostomy surgery is the need to move the ostomy site due to initial poor placement or herniation. This involves a second surgery that can be made more difficult due to abdominal adhesions, internal bowel restrictions and blood supply. It has been reported, in 2009 alone, that there were approximately 17,000 related revisions and/or herniation repair procedures.

As such, there is a desire to obtain a continent ostomy to prevent major complications and follow-up revision surgery.

### SUMMARY OF THE INVENTION

The present invention provides a 3D-structured implant that fits the shape of the interface between the intestine and the abdominal wall. The general configuration can be a funnel-like or device construct where the “funnel neck” can encapsulate and support the intestine and the larger “funnel top” can attach to and reinforce the abdominal wall.

The implant can consist of a structure of flexible members (e.g., patterned strut members or mesh) that can be designed to have specific mechanical properties in different areas of the implant. In one embodiment, the mesh construct can be configured to have an auxetic property of deformation (e.g., negative Poisson's Ratio). As such, the implant can appropriately react or adapt to abdominal pressure that pushes on the implant. The foreshortening of the intestinal portion of the implant causes a diameter reduction as well, in effect squeezing the intestine upon increased pressure. This can have the dual benefit of holding the intestine tighter during high abdominal pressure events to prevent herniation/prolapse, and also providing sphincter-like squeezing force to maintain continence during these stress or pressure events.

In other embodiments, the implant can be configured to provide a gentle squeezing force around the intestine by including specific properties into the implant structure by changing the overall diameter, strut width, strut angles, strut shape, and the like. Additionally, the funnel neck portion of the implant can be designed with a number of undulations to allow for preferential folding or collapsing of the implant diameter to help with the described squeezing action.

In still other embodiments, magnets or like features can be included with the implant. The magnets can be included to facilitate or provide additional force or actuation to close the implant funnel neck and aid in providing a continent ostomy. The magnets can be embedded in specifically designed cells of the implant structure, over-molded into the implant, or otherwise attached or provided with the implant. The distribution and configuration of the magnets with the implant can be designed to be both attractive and repulsive to achieve specific behaviors out of the implant. For example, the magnets can be attractive in a circumferential configuration to provide squeezing on the intestine. Alternatively, the magnets can have an attractive force in the longitudinal direction to rely on the auxetic nature of the implant to provide intestinal squeezing while still having a repulsive force circumferentially to prevent over-constriction of the intestine. A plug can be provided as well with magnets adapted to attract to the magnets of the implant to facilitate securement of the plug relative to the implant, with the intestinal wall provided therebetween.

Additional embodiments of the ostomy implant can include coatings to prevent adhesion or to facilitate healing. For instance, a coating of a methylcellulose compound on the external side of the intestinal portion of the mesh or struts could be included to prevent adhesion and maintain relative movement of the organ against adjacent tissue. Barbed or like anchor devices can be included to a periphery portion of the abdominal wall portion of the implant to speed up implantation and fixation.

Manufacture or formation of the implant can be accomplished by injection molding, direct extrusion rapid-prototyping, or via like processes or techniques.

### BRIEF DESCRIPTION OF THE DRAWINGS

FIGS. 1-2 are schematic views of ostomy implant devices in use, in accordance with embodiments of the present invention.

FIG. 3 is a schematic view of an ostomy implant device applying a level of constriction on the patient's intestine, in accordance with embodiments of the present invention.

FIGS. 3a-3c are partial views of patterned strut and cell configurations for use with portions of an ostomy implant device, in accordance with embodiments of the present invention.

FIG. 4 is a schematic view of an ostomy implant device in an initial or normal resting state, in accordance with embodiments of the present invention.

FIG. 5 is a schematic view of an ostomy implant device in a constriction or pressure state, in accordance with embodiments of the present invention.

FIG. 6 is a schematic top view of an ostomy implant device having a lobed region, in accordance with embodiments of the present invention.

FIG. 7 is a partial schematic view of an ostomy implant device portion including a magnet, in accordance with embodiments of the present invention.

FIG. 8 is a top schematic view of an ostomy implant device having magnets and an attraction polarity configuration, in accordance with embodiments of the present invention.

FIG. 9 is a top schematic view of an ostomy implant device having magnets and a repulsive polarity configuration, in accordance with embodiments of the present invention.

FIGS. 10-11 are schematic views of ostomy implant devices and plugs each having magnets, in accordance with embodiments of the present invention.

FIG. 12 is a schematic view of a plug having a venting feature, in accordance with embodiments of the present invention.

### DETAILED DESCRIPTION OF PREFERRED EMBODIMENTS

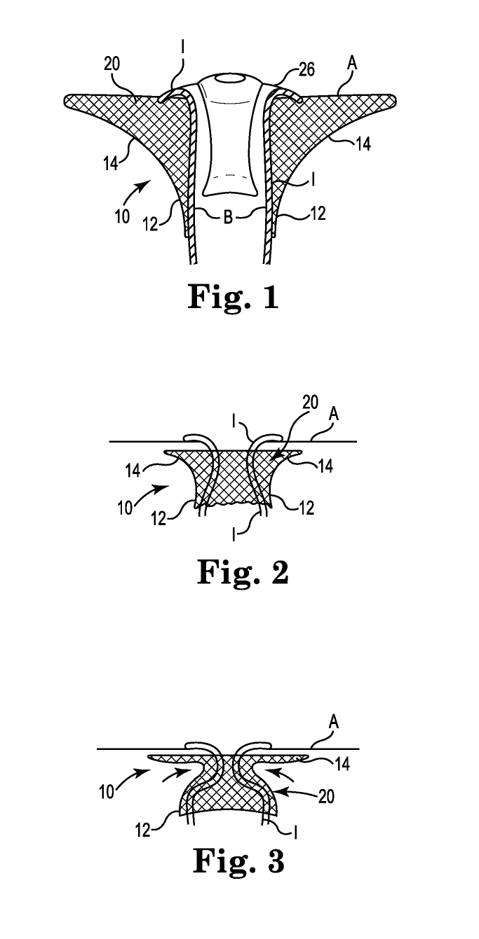

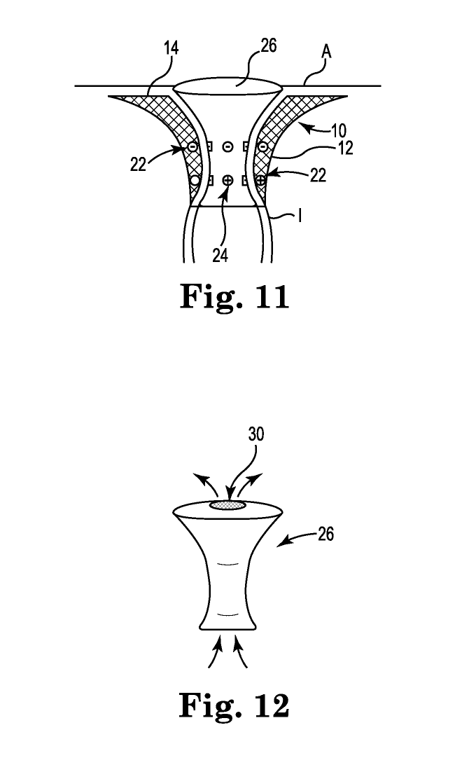

Referring generally to FIGS. 1-12, the present invention provides a 3D-structured implant that fits the shape of the interface between the intestine I and the abdominal wall A for ostomy or like procedures. The general configuration can include a funnel-like implant device 10 in which a funnel neck portion 12 can encapsulate and support a portion of the intestine and a generally larger funnel top portion 14 can attach to and reinforce the abdominal wall. In the case of a colostomy, a portion of the large intestine I is brought through and attached to the abdominal wall A, forming a stoma, to carry feces out of the body.

The implants 10, and portions thereof, could take on a myriad of different sizes, shapes and configurations depending on the particular treatment application, or deployment and support needs. The various implants 10, structures, features and methods detailed herein are envisioned for use with many known implant and repair devices (e.g., for male and female), features, tools and methods, including those disclosed in U.S. Pat. Nos. 7,500,945, 7,407,480, 7,351,197, 7,347,812, 7,303,525, 7,025,063, 6,691,711, 6,648,921, and 6,612,977, International Patent Publication Nos. WO 2008/057261 and WO 2007/097994, and U.S. Patent Publication Nos. 2011/0124956, 2011/0144417, 2010/0261955, 2002/151762 and 2002/147382. Accordingly, the above-identified disclosures are fully incorporated herein by reference in their entirety.

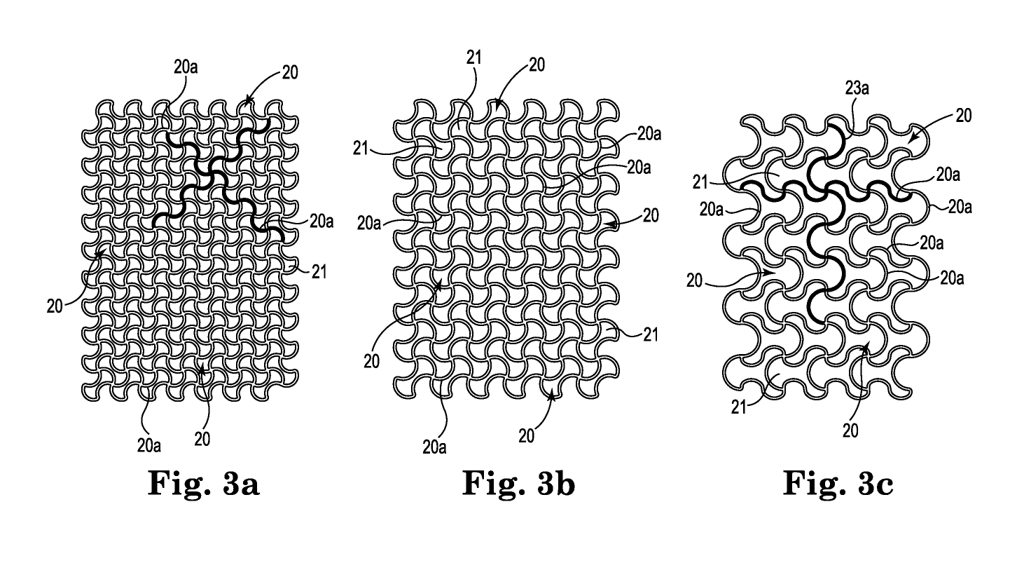

As shown in FIGS. 1-7, the implant 10 can consist of a structure of flexible members (e.g., patterned strut members) to create a cell construct 20 designed to have specific mechanical properties in areas of the implant (e.g., FIGS. 3-5). The various patterned strut configurations and support properties disclosed in the above-incorporated U.S. Patent Publication No. 2011/0144417 can be employed with the present invention. Various embodiments can be constructed of a polymer material such as polypropylene. Other embodiments can be constructed at least in part of a metal, such as Nitinol.

For instance, portions of the implant 10, such as portions 12, 14 can be formed or patterned by way of a polymer molding process to create a unitary homogeneous non-woven, or non-knitted, device or construct. Other embodiments can be formed from an already unitary homogeneous sheet or film via laser cutting, die cutting, stamping and like procedures. Still other embodiments can be constructed of woven or knitted mesh filaments where a non-homogenous mesh material is desired.

As a result of the manufacturing process, such as molding or cutting for homogenous or non-woven implants 10, repeating cells 20 form a lattice structure for at least the portions 12, 14 of the implant 10, as shown in FIGS. 3a-3c. These portions can be formed into sinusoid, or other waveform or undulating struts 20a to control elongation or compression along single or multiple axes, to define a desirable pattern density with overall reduced surface area, and to control the distribution and shaping from applied loads. The ability to mold, form or cut the struts 20a in a nearly endless array of sinusoidal or like configurations provides an implant 10 that can better provide support and avoid herniation.

In certain embodiments, the patterned struts 20a define a general pinwheel configuration to further define cellular voids 21 for the cells 20 (e.g., FIGS. 3a-3b). The thickness, size and separation of the struts 20a can be modified to create an implant 10 with different surface area and cellular density attributes.

The cross section of non-woven strut members 20a can be generally circular, oval or otherwise formed to have rounded portions with exemplary embodiments of the present invention. This can be an advantage over the bunched woven or knitted filament mesh strands of conventional implants. The rounded portions of the struts 20a of the present invention provide an improved implantation feel and a consistent surface adapted to retain its shape and to reduce or eliminate snagging or resistance during deployment and positioning. In addition, it provides a desirable tactile feel and surface for the physician to grasp and manipulate during implantation.

The implant 10, as generally shown in FIGS. 6 and 8-9, includes an interior receiving region or lumen 15 adapted to surround a portion of the intestine I. The implant 10 can be provided to the physician as a continuous circumference construct adapted to traverse along a portion of the intestine I to secure the intestine within the region 15. In certain embodiments, the implant 10 can be shaped to encompass or surround the intestine I by joining free ends or longitudinal edges of the implant 10. The implant 10 can be wrapped around the intestine I with the free ends attached or joined together (suture, clips, hooks, etc.) to secure the implant 10 around the intestine I. Further, the top portion 14 can be attached to the interior abdominal wall via suturing, stapling, adhesives, tissue anchoring and like surgical attachment devices and techniques.

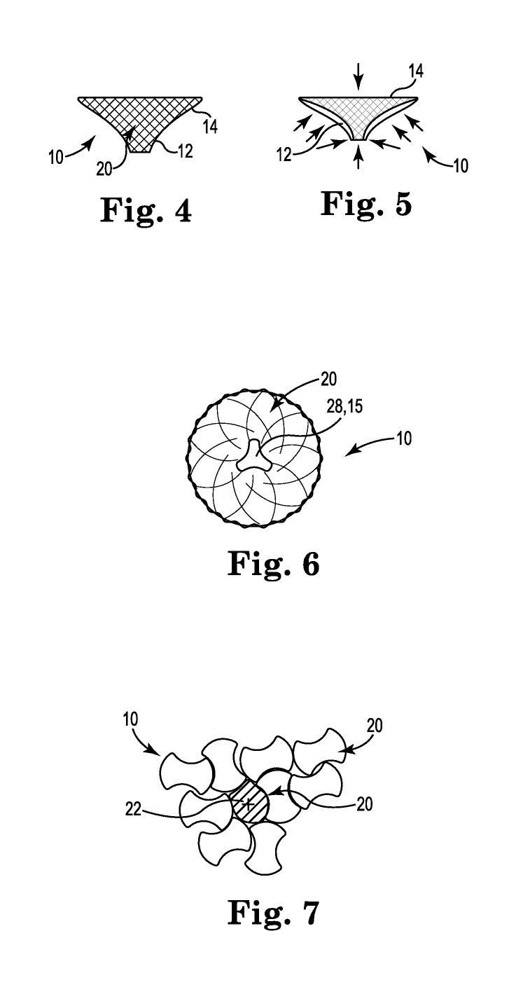

As shown in FIGS. 2 and 4, the implant can provide a level of gentle pressure or squeezing force on the intestine I to promote continence even in its normal state without high pressure events. Such a configuration promotes continence and assists in preventing herniation of the intestine I. The level of pressure, support and reaction to high-pressure events for the implant 10 can be controlled by changing the overall diameter of the funnel-shape, strut widths, strut angles, strut shapes, cell spacing and shapes, and the like. Additionally, the funnel neck portion 12 of the implant can be designed with a number of undulations to allow for preferential folding or collapsing of the implant diameter to help with the described squeezing action.

In certain embodiment, as demonstrated in FIGS. 3 and 5, the mesh construct 10 can be configured to have an auxetic property of deformation (e.g., negative Poisson's Ratio). Traditional knitted or woven mesh implants can tend to compress and narrow during longitudinal stretching, thereby displaying a positive Poisson affect or ratio. The combination of the struts and fixed junctions for the implant 10 of the present invention can facilitate a Negative Poisson affect such that the implant 10 can appropriately react or adapt to abdominal pressure that pushes on the implant. As shown in FIG. 5, the foreshortening of the intestinal portion 12 of the implant 10 causes a diameter reduction as well, in effect squeezing the intestine I upon increased pressure—e.g., from coughing, sneezing and like events. This can have the dual benefit of holding the intestine tighter during high abdominal pressure events to prevent herniation, and also providing sphincter-like squeezing force to maintain continence during these stress or pressure events.

FIG. 6 shows an embodiment of the implant 10 having a lobed portion 28 adapted to encapsulate or surround a portion of the intestine I. This lobed portion 28 creates hinging action for the implant 10 around the bowel, thereby providing a living or actionable sphincter. Such a construct can aid in collapsing the implant 10 around the bowel to provide better sealing of the bowel to promote continence.

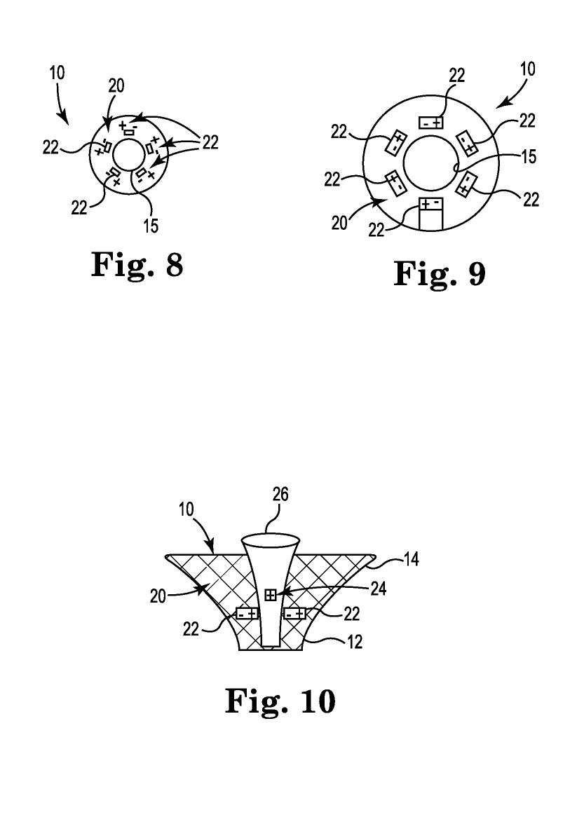

In still other embodiments, as shown in FIGS. 7-11, magnets 22 can be included with the implant 10. The magnets 22 can be included to facilitate or provide additional force or actuation to close the implant funnel neck 12 and aid in providing a continent ostomy. The magnets 22 can be embedded in specifically designed cells 20 of the implant structure 10 (e.g., FIG. 7), over-molded into the implant 10, or otherwise attached to or provided with the implant 10. The distribution and configuration of the magnets 22 with the implant 10 can be designed to be both attractive and repulsive to achieve specific behaviors out of the implant. For example, in certain embodiments the magnets 22 and the respective polarity can be attractive in a circumferential configuration to provide squeezing on the intestine I—e.g., FIG. 8. Alternatively, the magnets 22 can have an attractive force in the longitudinal direction to rely on the auxetic nature of the implant 10 to provide intestinal squeezing while still having a repulsive force circumferentially (e.g., FIG. 9) to prevent over-constriction of the intestine I.

The magnets 22 on the implant 10 can be configured to interface with an external device that can be inserted into the ostomy or on the outside of the ostomy, such as a plug 26 (FIGS. 10-12). The plug 26 can be configured with magnets 24 of its own and in a manner to cause attraction between the plug 26 and the implant 10, to facilitate entrapment of the intestinal wall I therebetween. This is beneficial as it relies on both the attractive sealing of the plug 26 to the interior intestinal wall I as well as collapsing of the intestinal lumen by the flexibility of the implant 10 structure around the outer wall of the intestine I. The interface options between magnets 22 in the implant 10 and magnets 24 in the plug 26 can be accomplished in various ways. In one embodiment, the magnet polarity in the plug 26 is in one direction with the opposite polarity being presented by the implant 10 magnets 22 to provide an attractive lock or securement configuration. Alternatively, the magnet polarity in both structures 10, 26 could have a pattern such that the interface between the implant 10 and the plug 26 is only facilitated in a certain (or a single) orientation. The magnet polarity in the implant 10 can be repulsive to itself in order to draw the opposing portions around the intestine I away to open the lumen for emptying, while the polarity of the magnets 24 in the plug 26 negate this repulsive force to allow collapsibility and attraction between the plug 26 and the implant 10 to close the intestinal lumen for continence when the plug 26 is inserted. The plug 26 can be or include a stoma bag for purposes of the various embodiments.

In certain embodiments, the magnets 22 are fixed or otherwise provided with the implant 10. In other embodiments, there can be some relative motion between the magnet 22 and the implant 10, e.g., within or along cells 20 or struts 20a. Further, the magnets 24 of the plug 26 can be selectively movable or actuated to move a distance away from the magnets 22 of the implant 10 to assist in moving the plug 26, releasing the plug 26, or adjusting the plug 26 relative to the implant magnets 22.

Additional embodiments of the ostomy implant 10 can include coatings to prevent adhesion or to facilitate healing. For instance, a coating of a methylcellulose compound on the external side of the intestinal portion 12 of the implant 10 can be included to prevent adhesion and maintain relative movement of the organ against adjacent tissue. Barbed or like anchor features can be included to a peripheral portion of the abdominal wall portion 14 of the implant to speed up implantation and fixation.

Manufacturing or formation of the implant 10 can be accomplished by injection molding, extrusion, rapid-prototyping, laser etching, or via like processes or techniques.

FIG. 12 shows an embodiment of the plug 26 having a gas-permeable membrane or like venting feature 30 to release flatus through the plug 26. This feature 30 can also serve to filter the odor escaping through the plug 26 as a result of the gas release. Further, the plug 26, e.g., within the lumen or cavity of the plug 26, can include a foldable bag or like device adapted to receive and contain the fecal contents from the bowel.

The implant systems 10, their various components, structures, features, tools, materials and methods may have a number of suitable configurations as shown and described in the previously-incorporated references. Various methods and tools for introducing, deploying, anchoring and manipulating implants to treat incontinence and prolapse as disclosed in the previously-incorporated references are envisioned for use with the present invention as well. Further, the system and its components or structures can be constructed of known and compatible materials know to those skilled in the art, including metals, polymers, and the like.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. A generally funnel-shaped ostomy implant, comprising:
a neck portion constructed at least in part of a plurality of undulating strut members, the neck portion adapted to surround a portion of the intestine of a patient; and
a top portion adapted to extend from the neck portion and attach to the abdomen of the patient.

2. The implant of claim 1, wherein the top portion is constructed at least in part of a plurality of undulating strut members.

3. The implant of claim 1, wherein the neck portion and the top portion provide a unitary construct of undulating strut members.

4. The implant of claim 1, wherein the top portion is attached to an interior surface of the abdomen.

5. The implant of claim 1, wherein the neck portion includes one or more magnets.

6. The implant of claim 5, wherein the one or more magnets are adapted to secure the neck portion around the intestine of the patient.

7. The implant of claim 1, further including a plug device adapted to insert into a portion of the intestine extending through the abdomen of the patient.

8. The implant of claim 7, wherein the plug device includes one or more magnets.

9. The implant of claim 8, wherein the neck portion includes one or more magnets adapted to cause attraction with the one or more magnets of the plug device.

10. An ostomy implant system, comprising:
a generally funnel-shaped implant including;
a neck portion constructed at least in part of a plurality of undulating strut members, the neck portion adapted to surround a portion of the intestine of a patient;
a top portion constructed at least in part of a plurality of undulating strut members and adapted to extend from the neck portion and attach to the abdomen of the patient; and
a plug device adapted to fit into an intestinal opening in the patient.

11. The system of claim 10, wherein the neck portion and the top portion provide a unitary construct of undulating strut members.

12. The system of claim 10, wherein the top portion is attached to an interior surface of the abdomen.

13. The system of claim 10, wherein the neck portion includes one or more magnets.

14. The system of claim 13, wherein the one or more magnets are adapted to secure the neck portion around the intestine of the patient.

15. The system of claim 14, wherein the plug device includes one or more magnets adapted to cause attraction with the one or more magnets of the neck portion.

16. An ostomy implant system, comprising:
a generally funnel-shaped implant including a neck portion and a top portion, the neck portion being constructed at least in part of a plurality of undulating strut members and having one or more magnets such that the neck portion is adapted to surround a portion of the intestine of a patient, with the top portion constructed at least in part of a plurality of undulating strut members and adapted to extend from the neck portion and attach to the abdomen of the patient; and
a plug device adapted to fit into an intestinal opening in the patient, the plug device having one or more magnets adapted to cause attraction with the one or more magnets of the neck portion.

17. The system of claim 16, wherein the neck portion and the top portion provide a unitary construct of undulating strut members.

18. The system of claim 16, wherein the top portion is attached to an interior surface of the abdomen.

19. The system of claim 16, wherein the undulating strut members define a plurality of repeating cells.

20. The system of claim 19, wherein the plurality of repeating cells are generally pin-wheeled shaped.
